SEQUENCE LISTING (1 sequence)

SEQ ID NO: 1 - protein, 13 aa, Artificial Sequence
  Description of Artificial Sequence Synthetic Peptide
SVRIGLPKEESRN

(19) United States
(12) Patent Application Publication
(10) Pub. No.: US 2005/0005311 A1
(43) Pub. Date: Jan. 6, 2005

(54) High throughput screening for cancer genes

(76) Inventors: Lance A. Liotta, Bethesda, MD (US); Elizabeth Woodhouse, North Potomac, MD (US); Emanuel Petricoin, Dunkirk, MD (US)

Correspondence Address: EDWARDS & ANGELL, LLP, P.O. BOX 55874, BOSTON, MA, 02205, US

(21) Appl. No.: 10/835,549

(22) Filed: Apr. 28, 2004

Related U.S. Application Data
(60) Provisional application No. 60/466,089, filed on Apr. 28, 2003.

(51) Int. Cl. A01K 67/033; C12Q 1/68

(52) U.S. Cl. 800/8; 435/6

(57) ABSTRACT

The invention provides high throughput screening systems and in vivo methods for high throughput screening of cancer genes. The invention also is applicable to the discovery of therapeutic agents that block tumor growth and metastasis. The invention further provides kits and compositions to perform such assays.

### FIELD OF THE INVENTION

The invention relates to high throughput screening systems for identifying genes causing abnormal cellular proliferation and for identifying agents that modulate the expression and/or activity of these genes. In particular, the invention relates to a whole organism-based assay system to identify cancer genes and modulators thereof.

### BACKGROUND

Cancer metastasis is a complex multi-step process involving numerous signaling pathways. In the past, the involvement of particular genes in metastasis has been inferred from correlation studies, in which the genome of patients who have cancer or who are at risk for cancer has been screened for alterations that might be linked to the phenotype of abnormal cellular proliferation. However, because of the complex genetic interactions involved in metastasis, it has been difficult to distinguish molecules which are functionally required for this lethal process from those which are incidental and downstream.

High throughput screening assays (“HTS assays”) that are cell-free or cell-based have been used to attempt to scan the genome for genes that are implicated in abnormal cellular proliferation. Such genes and their gene products are likely to represent targets for drug development. Similarly, HTS assays are being used in drug screening protocols based on identified targets to assay large numbers of compounds for putative biological activity (e.g., inhibition or activation of a particular target).

Current HTS assays have limited ability to identify genes that are functionally involved in abnormal cellular proliferation processes, such as cancer. In the case of cell-free assays, screening is typically limited to assaying for proteins that interact with single candidate target molecules and requires extrapolating back to the genes that encode such proteins. Such screens typically attempt to identify proteins that modulate the activity of cell signaling proteins.

In contrast, cell-based assays permit screening for gene products that interact with a target expressed in a cell. The readouts of such assays can include the physiological properties of the cell, such as differentiation and/or proliferation, and thus necessarily have more biological relevance. However, even cell-based assays are hampered by the artificial context of cells replicating outside of a biological organism in which a cancer phenotype is ultimately expressed.

HTS assays for relevant lead drugs suffer from similar artifacts. It is difficult to predict from cell-free assays, how a lead will interact with other molecules and gene products in a cell, much less an entire organism. While cell-based assays provide the potential to make initial determinations regarding bioavailability, they often provide inadequate similarity to an in vivo disease condition, since most diseases develop within multicellular tissues.

The genetic manipulation of a whole organism to identify genes involved in abnormal developmental processes has been exemplified in the study of the fruit fly, Drosphila melanogaster. Homeotic genes constitute one of the best-known examples of genes first identified in Drosophila that have provided insight into the mechanisms of human development and disease (see e.g., van Heyningen, Mol. Med 3: 231-237, 1997).

U.S. Pat. No. 6,316,690 reports screening flies that contain a v-myb transgene, a gene not normally expressed in flies, by feeding larvae or adult flies a candidate drug compound and screening for a change in neoplastic phenotype. The patent discloses screens for spontaneous development of tumors in larval stages of Drosphila.

PCT publication WO 01/51604 reports transgenic flies containing recombinant sensitizer genes that are mutated or misexpressed in a way that increases abnormal cell proliferation but which do not cause lethality or infertility. WO 01/51604 describes using recombinant constructs providing for tissue-specific expression of a human oncogene or cell cycle gene to create such a phenotype. The publication also describes using transposon mutagenesis to screen for “interactor” genes that enhance or suppress abnormal cell proliferation in the presence or absence of one or more tumor agents. There is an inherent selection bias for tissue-specific cell growth factors in this system and for mutations that are cell lethal or dominant.

WO 00/37938 discloses screening for small molecule modulators of biochemical pathways by microinjecting candidate small molecule compounds into the open circulatory system of genetically modified Drosophila larvae which express a human gene involved in a signaling pathway. The publication also reports genetic screening for suppressors or enhancers of mutated Drosophila signaling genes.

### SUMMARY OF THE INVENTION

The invention provides a whole-organism based assay for identifying genes that are associated with tumorigenesis and metastasis. Preferably, the whole organism is small and multicellular with a rapid generation time and comprises multiple germ layers. More preferably, the organism comprises a high degree of conservation of the various signaling pathways involved in the etiology of human disease; can be grown rapidly in large numbers and comprises genetically mapped marker genes to facilitate mapping of newly identified mutations.

In particular, the invention provides an HTS system for identifying genes whose function is required for normal cellular proliferation and/or differentiation processes. The system exploits the rapid growth and well-characterized genetics of Drosophila melanogaster.

The high degree of conservation of morphogenetic processes between Drosophila and humans makes Drosophila a powerful system to use to screen, identify and characterize molecules that are functionally required for cellular invasion during cancer and metastasis. The components of signaling pathways between Drosophila and humans are also highly conserved.

In one aspect, the invention provides a method for identifying a gene that produces or modulates a neoplastic phenotype. The method comprises introducing a neoplastic tissue expressing a reporter sequence in an adult fruit fly. Preferably, the tissue is derived from a fly comprising a mutated gene whose expression, or lack of expression, results in non-tissue specific abnormal cell proliferation. Preferably, the adult fly expresses a gene, or can be induced to express a gene, that is altered (e.g., by a mutation) in a way that modulates the pattern of abnormal cell proliferation observed. For example, the altered gene modulates tumor induction (e.g., tumorgenicity, or numbers of tumors), tumor growth (e.g., numbers of cells in a tumor or tumor size) and metastasis (invasion into different tissues).

The presence or expression of the reporter sequence in cells from a plurality of different tissues in the adult fly is evaluated and one or more of: a change in the numbers of different tissues expressing the reporter sequence and a change in the quantity of the reporter sequence, in one or more tissues, identifies the presence of one or more mutated genes in the adult fly which are functional modulators of the neoplastic phenotype. In one aspect, the gene is a mutated Drosophila gene.

Preferably neoplastic tissue is obtained from the larval stage of a fly comprising a gene whose disruption is associated with the production of metastatic and invasive tumors. Deletion of the Drosophila gene lethal giant larvae, (l(2)gl), on the second chromosome, leads to highly invasive and widely metastatic tumors upon transplantation into adult flies. By mutagenizing flies with an l(2)gl genetic background, modulator mutations can be selected for which alter the neoplastic phenotype associated with the l(2)gl mutation. For example, tissue from larvae which are homozygous for the modulator mutation and the l(2)gl mutation can be evaluated for neoplastic potential by introducing the tissue into adult flies comprising functional l(2)gl genes and modulator genes.

Preferably, mutations are generated at random, allowing the entire genome to be scanned for potential modulator genes. More preferably, mutations are generated using P-elements comprising markers that can be used to select for viable homozygotes bearing two copies of a mutated gene. The proliferation of l(2)gl cells in such flies can be tracked by assaying various cells, tissues, or body segments, of the adult fly for the expression of the reporter gene expressed by the neoplastic cells. In one aspect, the P-element comprises both the marker gene and the reporter sequence. This assay allows for quantitative and qualitative measures of abnormal cell proliferation in the flies being screened.

Mutated genes can be readily cloned using the P-elements as tags for these genes.

Mapping is simplified by the well-developed cytogenetic and molecular analyses permitted by Drosophila. The functional role of the gene can be verified using P-element mediated rescue to introduce wild-type copies of the gene back into the fly and/or to monitor the effect of excision of P-elements from a particular gene.

In an HTS assay according to a second aspect of the invention, l(2)gl neoplastic tissue comprising a reporter gene is introduced into an adult fly comprising a functional l(2)gl gene, and a candidate modulator of a neoplastic gene is introduced into the nutrient medium on which the fly (or a larval form thereof) feeds. The ability of the modulator to alter the pattern of tumor growth in the fly is assessed. The proliferation of neoplastic cells, such as l(2)gl cells, is tracked by detecting the presence (e.g., expression and/or activity) of a reporter gene expressed in the neoplastic cells in various cells, tissues and/or body segments of the adult fly. This assay allows for quantitative and qualitative measures of abnormal cell proliferation in the flies being screened. In one aspect, the candidate modulator is a candidate therapeutic agent that decreases tumorgenicity or metastasis. However, in another aspect, the screen is used to evaluate the carcinogenic potential of an agent.

The invention further provides compositions and kits. In one aspect, a kit comprises an array comprising a substrate, such as a polymer, nitrocellulose, glass, silicon, and the like. Samples comprising a plurality of different cellular polypeptides and/or nucleic acids are obtained from a mutant fly comprising a mutated modulator gene identified as described above. The samples are arrayed at different locations on the substrate (e.g., using an automatic microarrayer as described above).

In one aspect, the samples comprise extracts from one or more cells from larvae of the mutant fly strain comprising the mutated modulator gene. The fly strain comprising the mutated modulator gene may also be mutated for one or more copies of a tumorigenic gene. The arrays can be packaged into kits. Such kits may further comprise at least one molecular probe such as an antibody or nucleic acid. Preferably, the probe is labeled. More preferably, the probe specifically binds to a molecular pathway molecule, such as a cell signaling protein. In a further aspect, at least one probe in the kit recognizes a modified form of a polypeptide but does not recognize an unmodified form of the polypeptide.

The invention also provides a composition comprising one or more isolated neoplastic cells from Drosophila. For example, the composition comprises one or more cells comprising a mutation in a tumorigenic gene and expressing a neoplastic phenotype (e.g., the cells are homozygous or hemizygous for a recessive mutation, or are heterozygous or homozygous for a dominant mutation). Tumorigenic genes include, but are not limited to, l(2)gl, brat, (l(3)bt), l(3)mbt, Dlg, tu (2)-K, and e(tu-K).

Preferably, the one or more cells are from one or more larvae. Also, preferably, the one or more cells comprise a reporter sequence. The reporter sequence may be selected from any of the sequences described above. Preferably, the reporter sequence is comprised within a P-element.

In one aspect, the one or more cells are frozen.

The kit may additionally comprise one or more of the compositions described above and one or more reagents for facilitating injection of the one or more cells into an adult fly.

The one or more cells additionally may comprise at least one mutation in a modulator gene.

A number of advantages are provided by HTS systems according to the invention. The HTS assays according to the invention can be used to screen large populations of flies (e.g., greater than 100,000) to identify candidate genes or agents that affect abnormal cellular proliferation. Because screening is performed in adult flies, the screens for mutated genes select for genes that are adult viable. Thus, a link to tumorigenicity and/or metastasis will not simply be due to a constitutive role for the gene in normal development and morphogenesis. Further, the screening systems rely on the use of a mutation in a gene naturally found in Drosophila, l(2)gl. Thus, the phenotypic impact of the mutation is based on the perturbation of a gene product that normally interacts with other Drosophila cellular proteins. The restoration of a normal phenotype in l(2)gl flies is therefore more likely to reflect biologically relevant modulators of cell proliferation which may have counterparts in mammals, particularly human beings. Similarly, the HTS assays evaluate neoplastic phenotypes in adult flies, rather than in larvae, whose cells cycles are adapted to the unique constraints of metamorphosis. Because there is no tissue-specific bias to the oncogenic potential of the cells being tested, the HTS assays according to the invention are less likely to impose a selection bias for modulators that have unique effects in specific tissue types.

### BRIEF DESCRIPTION OF THE FIGURES

The objects and features of the invention can be better understood with reference to the following detailed description and accompanying drawings.

FIGS. 1A-H illustrate a functional screen for metastasis genes according to one aspect of the invention. FIG. 1A is a schematic diagram showing the use of P-element mutagenesis of a Drosophila genome heterozygous for a mutation in l(2)gl to scan the genome for mutations which are modulators of the neoplastic phenotype of l(2)gl. Adults homozygous for a P-element and heterozyogus for l(2)gl deletion are crossed to generate larvae that are homozygous l(2)gl and homozygous for P-element insertion. Brain tissue from these larvae is transplanted into adults. FIGS. 1B-H show metastasis patterns of l(2)gl insertion, and excision lines (described further below).

FIGS. 2A-D are as described below.

FIG. 2A is a schematic illustrating cloning of genomic regions flanking P-element insertion sites. Genomic regions from P-element insertion lines are indicated with arrowheads at P-element insertion site. The 97-2 insertion is located 15.6 kb from the Pi3K59F gene and 16.3 kb from the apontic gene. The 115-1 insertion is 445 bp from the start of the translated region of the pointed gene while the 23-2 insertion is 46 by from the start of the translated region of the semaphorin 5c (sema-5c) gene. FIGS. 2B-C show expression analysis of three modulator genes identified using the HTS system according to one aspect of the invention. FIG. 2B shows PCR amplification of the 23-2 insertion with primers specific for 3′P-element sequence and genomic sequence flanking the 23-2 insertion. Lane 1: parental genomic DNA, tubulin primers; lane 2: parental genomic DNA, 23-2 insertion primers; Lane 3: 23-2 genomic DNA, tubulin primers; lane 4: 23-2 genomic DNA, 23-2 insertion primers. The tubulin PCR product 655 bp. The 23-2 PCR product is 241 bp. FIG. 2C shows RT-PCR analysis of apontic gene expression. Lane 1: Parental line cDNA, tubulin primers; Lane 2: parental line cDNA, apontic primers; Lane 3: 97-2 cDNA, tubulin primers; Lane 4: 97-2 cDNA, apontic primers. The apontic RT-PCR product is 174 bp. The tubulin RT-PCR product 165 bp. FIG. 2D shows RT-PCR analysis of pointed expression. Lane: 1 115-1 cDNA, tubulin primers; 115-1 cDNA. Lane 2: pointed primers; Lane 3: parental line cDNA, tubulin primers; Lane 4: parental line cDNA, pointed primers. The tubulin RT-PCR product is 165 bp. The ets-like RT-PCR product is 129 bp.

FIGS. 3A-C shows restoration of a neoplastic phenotype by reintroduction of the wild-type modulator gene, sema-5c, into l(2)gl homozygotes. FIG. 3A shows Western blotting of Drosophila brain extracts with anti-semaphorin antibodies. The parental line expresses sema-5c (lane 1). The 23-2 insertion line lacks sema-5c expression (lane 2). The 23-2 excision line restores sema-5c expression (lane 3). FIG. 3B is a schematic diagram showing Class 5 semaphorin domains. FIG. 3C shows protein microarray analysis of selected signaling proteins in l(2)gl/l(2)gl and l(2)gl/l(2)gl sema-5c/sema-5c brain tissues. Wild-type values were subtracted from l(2)gl/l(2)gl and l(2)gl/l(2)gl sema-5c/sema-5c values.

FIGS. 4A to B show that SEMA5A protein expression correlates with metastatic potential in murine and human tumor cell lines. FIG. 4A shows Western blot analysis of SEMA5A and P-SMAD1 in 3T3 cells transfected with indicated constructs: Ras+ATX (highly metastatic), Ras (metastatic); Mock-transfected 3T3 cells (non-metastatic). SEMA5A expression was compared in human tumor cell lines: MDA435 (highly metastatic); MDA231 (low metastatic potential), A2058 (non-metastatic). FIG. 4B shows immunostaining of MDA 435 cells with semaphorin antibodies, verifying a cell membrane localization of SEMA5A.

FIG. 5 is a bar graph illustrating that the P13K inhibitor, LY294002, blocks l(2)gl primary tumor growth in Drosophila but an ERK inhibitor, PD98059, does not. Adult hosts injected with l(2)gl/l(2)gl larval tissue were orally administered drugs for 21 days after injection. Hosts were treated with 0 or 0.56 μg/ml of LY294002 (reduction of tumor size to 7% of untreated) and 0 or 0.56 μ/ml PD98059 (no effect).

FIG. 6 (A and B) show the expression of Dpp target gene vestigial is increased in l(2)gl brain tissue compared with wild-type. RT PCR analysis demonstrated elevated vestigial levels (quantitated in proportion to tubulin) (n=3) in l(2) gl tissues compared with wild-type or lgl/lgl; sema-5c/sema-5c. FIG. 6 (c) shows a model for the role of TSP-1 repeats in Semaphorin 5c activation of the Dpp pathway.

FIG. 7 shows the expression of human homologs of Semaphorin 5C, including KIAA 1445 (Sema 5D). FIG. 7A shows the expression of SEMA5A and SEMA5D being detected in membrane preparations of A2058 human melanoma cells. FIG. 7B shows the results of an immunohistochemistry assay, which demonstrates membrane localization of SEMA5D in ovarian cancer cells.

### DETAILED DESCRIPTION

The invention provides high throughput screening systems and in vivo methods for high throughput screening of cancer genes. The invention also is applicable to the discovery of therapeutic agents that block tumor growth and metastasis.

Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

The term “a fly”, unless the context indicates otherwise, generally refers to any stage of a fly's development (e.g., embryo, larva, pupa, adult) and may further refer to a population of flies. When referring to a population of flies, the term “fly” preferably refers to a substantially isogenic population of flies. The term “fly”, “population of flies” and “fly strain” may be used interchangeably in certain contexts.

As used herein monitoring the expression of a reporter sequence in “cells from a plurality of different tissues” refers to monitoring the presence of and/or expression of the reporter sequence and/or monitoring the activity of a reporter sequence product (e.g., such as a protein or transcript). The cells do not need to be isolated from the fly and can be monitored in situ. The term “plurality” refers to at least two.

The term “proliferation” as used herein means growth and division of cells.

As used herein, the term “normal cells” refers to cells that have a limitation on growth, i.e., a finite number of division cycles.

The term “abnormal cellular proliferation” refers to one or more of a: a removal on a limitation on growth, an inability to remain within appropriate cell boundaries, de-differentiation, and an increase in size in a group of cells at a target site (e.g., a tumor site) which has no normal physiological function.

As used herein, a cell with a “neoplastic phenotype” refers to a phenotype of abnormal, uncontrolled cellular proliferation. Neoplastic cells have a greater ability to cause tumors when injected into a host multicellular organism. A neoplastic phenotype can be recognized by changes in growth characteristics, particularly in requirements for growth factors, and often also by changes in morphology. Neoplastic cells usually proliferate without requiring adhesion to a substratum and usually lack cell to cell inhibition. Neoplastic cells tend to show partial or complete lack of structural organization and functional coordination with the normal tissue, and may be benign or malignant. A neoplastic phenotype may be determined by the induction of at least one tumor in a host organism upon the introduction of cells having a “neoplastic phenotype”.

As used herein, “a tumorigenic gene” is a gene whose disruption results in a neoplastic phenotype. A disruption may be an alteration of gene expression and/or an alteration of the activity of a gene product.

As used herein, a “modulator mutation” refers to a mutation in a “modulator gene” which, when disrupted, alters the neoplastic phenotype of a tumorigenic gene. In one aspect, a modulator causes a significant change in one or more of the numbers of tumors induced in a single organism or in a population of organisms, the size of tumors (e.g., numbers of cells which are proliferating abnormally), and/or which changes the amount of metastasis observed, as determined using routine statistical tests, setting p <0.05, or about <0.01. In one aspect, a modulator changes the size of a tumor by at least about 10%. In another aspect, a modulator changes the size of a tumor by at least about 2-fold. In a further aspect, a modulator changes the number of cells proliferating abnormally by at least about 10% or at least about 2-fold. In still another aspect, a modulator alters the amount of metastasis (e.g., as determined by the number of neoplastic cells observed in areas distal to an injection site, or by the numbers of neoplastic cells in different tissue types) by at least about 10% or at least about 2-fold. A “suppressor of a neoplastic phenotype” or a “suppressor of a tumorigenic gene” causes a significant decrease in one or more of: the numbers of tumors induced in a single organism or in a population of organisms, the size of tumors, and/or which decreases the amount of metastasis observed, as determined using routine statistical tests, setting p <0.05, or about <0.01. An “enhancer of a neoplastic phenotype” or a “enhancer of a tumorigenic gene” causes a significant increase in one or more of the numbers of tumors induced in a single organism or in a population of organisms, the size of tumors (e.g., numbers of cells which are proliferating abnormally), and/or which increases the amount of metastasis observed, as determined using routine statistical tests, setting p <0.05, or about <0.01.

As used herein, “inhibiting cellular proliferation” refers to slowing and/or preventing the growth and division of cells.

The term “inhibiting metastasis” refers to slowing and/or preventing metastasis or the spread of neoplastic cells to a site remote from a primary growth area.

The term “invasion” as used herein refers to the spread of cancerous cells to surrounding tissues.

As used herein “a growth inhibitory amount” of a modulator compound is an amount capable of inhibiting the growth of a cell, especially a cell with a neoplastic phenotype. In one aspect, a growth inhibitory compound is one which significantly reduces the percentage of the target cells in anyone or all of the cell cycle phases, including G₀, G1, S phase, G2 and mitosis.

As defined herein, “homologous” refers to sequences that are at least about 60% identical, at least about 70% identical, at least about 75% identical, at least about 80% identical, at least about 90% identical, at least about 100% identical to a reference sequence. To determine the percent identity of two amino acid sequences or of two nucleic acid sequences, the sequences are aligned for optimal comparison purposes (e.g., gaps are introduced in one or both of a first and a second amino acid or nucleic acid sequence for optimal alignment and non-homologous sequences can be disregarded for comparison purposes). The percent identity between the two sequences is a function of the number of identical positions shared by the sequences, taking into account the number of gaps, and the length of each gap which need to be introduced for optimal alignment of the two sequences. The amino acid residues or nucleotides at corresponding amino acid positions or nucleotide positions, respectively, are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

A “comparison window” refers to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. For example, the percent identity between two amino acid sequences can be determined using the Needleman and Wunsch algorithm (J. Mol. Biol. 48: 444-453, 1970) which is part of the GAP program in the GCG software package (available at http://www.gcg.com), by the local homology algorithm of Smith & Waterman (Adv. Appl. Math. 2: 482, 1981), by the search for similarity methods of Pearson & Lipman (Proc. Natl. Acad. Sci. USA 85: 2444, 1988) and Altschul, et al. (Nucleic Acids Res. 25(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection. Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Exemplary gap weights using a Blossom 62 matrix or a PAM250 matrix, are 16, 14, 12, 10, 8, 6, or 4, while exemplary length weights are 1, 2, 3, 4, 5, or 6. The GCG software package can be used to determine percent identity between nucleic acid sequences. The percent identity between two amino acid or nucleotide sequences also can be determined using the algorithm of E. Myers and W. Miller (CABIOS 4: 11-17, 1989) which has been incorporated into the ALIGN program (version 2.0), using a PAM120 weight residue table, a gap length penalty of 12 and a gap penalty of 4.

As used herein, a “differentially expressed” gene product, as used herein, refers to a gene transcript or protein that is found in significantly different numbers of copies, or in activated versus inactivated states, in different cell or tissue types of an organism having a tumor or cancer, compared to the numbers of copies or state of the gene product found in the cells of the same tissue in a healthy organism, or in the normal cells of the same tissue in the same organism, as determined using routine statistical methods known in the art (e.g., setting p<0.05, or <0.01).

Whole-Organism Based Screening Assay

The invention exploits the rapid generation time and well-characterized genetics of Drosophila melanogaster to identify modulators of a mutation associated with the development of highly invasive and widely metastatic tumors in adult flies. In one aspect, the mutation causes abnormal cell proliferation in tissues. Preferably, the gene is homologous to a human gene.

Mutations Associated With Neoplastic Phenotypes

Flies which comprise a mutation in a gene associated with a neoplastic phenotype (“tumorigenic mutation”) are bred to homozygosity or otherwise exposed to conditions in which the phenotype is expressed to provide a source of neoplastic cells. Preferably, the mutation is highly penetrant, and highly expressed.

In one aspect, the mutation is a deletion at the locus on the second chromosome lethal (2) giant larvae (l(2)gl) at cytogenetic locus 21A2. The protein encoded by the l(2)gl gene is a myosin binding protein which is expressed in multiple tissues in embryos, in larval salivary glands, imaginal discs, ovary and brain, and in the heads of adult flies. Homologous sequences have been identified in Caenorhabditis elegans, mice, and humans. Amorphic mutations or loss of function mutations are recessive late lethal mutations that die predominantly as larvae, displaying a tumorigenic phenotype. When isolated l(2)gl neoplastic cells from imaginal discs and brain tissue are transplanted into adult flies, they metastasize rapidly upon transplantation into wild-type adult flies.

The l(2)gl protein is expressed in the cytoplasm and at regions of cell junctions on the inner face of the cell membrane (Strand, et al., J. Cell Biol. 127 (5), 1345-1360. 1994a). The protein is required, along with the tumor suppressors, discs large and scribble (Bilder, et al., Science 289: 113-116, 2000) for basal protein targeting (Peng, et al., 2000) and asymmetrical divisions of neuroblasts (Oshiro, et al., Nature 408: 593-596,2000). The l(2)gl protein is present in a high molecular weight protein complex, consisting primarily of l(2)gl homo-oligomers and the non-muscle myosin heavy chain (Strand, et al., J. Cell Biol. 127 (5): 1345-1360.1994b). The l(2)gl protein appears to promote basal protein targeting while myosin II is inhibitory to this process (Peng et al., Nature 408: 596-600, 2000). Homologs of l(2)gl exist in other species, including mouse (Tomotsune, et al., Nature 365: 69-72, 1993) and human (Strand, et al., Oncogene 11: 291-301, 1995) and are also associated with nonmuscle myosin (Strand, et al., J. Cell Biol. 127 (5), 1361-1373), so it is likely that the role of l(2)gl in maintaining cytoskeletal architecture is conserved. Homologs of genes that control metastasis in Drosophila may play a similar role in higher organisms.

Other genes with mutations conferring a tumorigenic phenotype also can be used as sources of neoplastic tissue. For example, certain mutant alleles of the brain tumor gene, brat, (brat¹, brat¹¹, brat¹⁴, brat^{fs3}) are associated with a tumorigenic phenotype. The brat gene, at 37B9 on chromosome 2, encodes a product involved negatively regulating the level of rRNA. Homologous sequences have been found in humans. Hemizygous larval brain tissue from brat¹¹ flies shows unrestrained and invasive growth when transplanted into the abdomens of adult female hosts. See, e.g., Wright, J. Hered. 87(): 175-190, 1996. Additionally, cells from transplanted brat¹¹/brat¹⁴ brain fragments form at least one secondary tumor in the wild-type host in 84% of cases while imaginal discs from brat¹¹/brat¹⁴ larvae form secondary tumors in 53% of hosts (Woodhouse, et al., Dev. Genes Evol. 207(8): 542-550, 1998).

The Drosophila gene lethal brain tumor, l(3)bt, has an allele (l(3)bt¹) which has a conditional (i.e., temperature sensitive) tumorigenic phenotype. At 29° C., third instar larvae develop brain tumors and die either before or after pupation. Brain tissue will grow in a malignant fashion when transplanted into wild type hosts. The temperature-sensitive period is 0-12 hours of embryonic life. See, e.g., Potter et al., Trends Genet. 16(1): 33-39, 2000.

The gene lethal (3) malignant brain tumor, l(3)mbt, which maps to 97E6-7, encodes a nuclear transcription factor which also is homologous with human sequences. The gene contains a sterile α motif (“SAM”)/Pointed domain. Mutations are recessive tumorigenic. For example, homozygous larvae of l(3)mbt^{E2}, l(3)mbt^{P3}, l(3)mbt^{ts1}, and l(3)mbt^{unspecified}, all develop brain tumors. Brain fragments of l(3)mbt^{ts1} and l(3)mbt^{unspecified} homozygous larvae reared at 29° C., when transplanted into wild-type flies, produce malignant tumors which invade the fat body, gut, thoracic muscles and the head, typically causing death of the host fly in 10-14 days.

The discs-large gene (Dlg) encodes a membrane-associated protein which has guanylate kinase activity. Similar sequences have been identified in humans. The gene is located on the X chromosome and maps cytologically to 10B13-14. Amorphic mutations are recessive tumorigenic but tend not to form secondary tumors on transplantation.

Combinations of mutant genes can be used to generate sources of neoplastic tissue. For example, while the allele tu (2)-K is associated with a poorly penetrant tumorigenic phenotype. Homozygous mutations of e(tu-K)¹ produce a significant increase in the penetrance of tu (2)-K¹ in both untreated flies and those treated in ways known to increase tumor incidence in tu (2)-K¹ (i.e., by suboptimal balances of pentose nucleotides, cholesterol deficiency, or an excess of L-tryptophan in the larval diet as well as by X irradiation of embryo).

Reporter Sequences

Preferably, flies homozygous for a tumorigenic mutation comprise at least one copy of a reporter sequence, allowing neoplastic cells obtained from these flies to be traced in a host adult fly into which they are transplanted. A reporter sequence preferably encodes a gene product whose level or activity can be easily measured in an HTS assay.

In one aspect, a reporter sequence is operably linked to a transcriptional regulatory element which is capable of driving expression of the reporter sequence in transplanted neoplastic cells. The product of the reporter sequence may be visually detectable, either in a fluorescence assay or after interacting, directly or indirectly, with a chromogenic substrate. Examples of such reporters include, the lacZ protein (P-galactosidase), green fluorescent protein (GFP), alkaline phosphatase, horseradish peroxidase, blue fluorescent protein (BFP), and luciferase photoproteins such as aequorin, obelin, mnemiopsin, and berovin (see, e.g., U.S. Pat. No. 6,087,476).

However, a reporter sequence may also be any nucleic acid sequence that is not found in the host fly and which may be detectable by a suitable assay (e.g., such as by PCR). Similarly, a reporter sequence can encode an antigenic sequence (e.g., a peptide) not typically expressed in the host cell, allowing neoplastic cells to be recognized by using antibodies to detect expression of the antigenic sequence. Commonly used and commercially available epitope tags include sequences derived from, e.g., influenza virus hemagglutinin (HA), Simian Virus 5 (V5), polyhistidine (6×His), c-myc, lacZ, GST, and the like. Antibodies specific to these epitope tags are generally commercially available. The expressed reporter can be detected using an epitope-specific antibody in an immunoassay or by FACs analysis.

Examples of suitable transcriptional regulatory elements include the Alcohol dehydrogenase (ADH) gene promoter, hsp 70 promoter, hsp 82 promoter, and the like. Reporter sequences can be integrated into the Drosophila genome using methods known in the art, such as P-element transformation, using the presence of a marker gene to follow the inheritance of the P-element. Suitable marker genes include white and rosy which affect eye color. Other marker genes in Drosophila include, but are not limited to, yellow, ebony, singed, and Mwh, which are body color or morphology markers. A comprehensive list of markers for Drosophila may be found in Ashbumer (In D. melanogaster: A Laboratory Manual, (1989) Cold Spring Harbor, N.Y., Cold Spring Harbor Laboratory Press: pp. 299-418).

Generation of Homozygous Mutations that Disrupt Metastasis

Random mutations are generated in a background that is heterozygous for the tumorigenic mutation to select for modulator genes whose mutation will disrupt metastasis (“modulator mutations”). Preferably, the tumorigenic mutation is a deletion rather than a mutation that produces an abnormal protein, to avoid selection for second site mutations at the tumorigenic gene locus. Modulator mutations can comprise insertions, deletions, point mutations, or rearrangements and can be induced using chemical agents, or exposure to x-rays or ultraviolet irradiation. However, preferably, modulator mutations are of a form that facilitates identification and cloning of the modulator gene. Therefore, in one preferred aspect according to the invention, modulator mutations are generated by insertion of a transposable element, such as a P-element.

P-elements comprise sequences recognizable by a transposase that enables the P-elements to be inserted into or removed from the genome. In fly strains expressing repressors of the transposase, the P-elements do not excise and are stably integrated in a fly's genome. When crossed to a fly strain lacking such repressors, P-elements will “hop” and insert at different genomic locations and can disrupt gene function when they land in a gene. By crossing back to a strain that comprises repressors, the newly inserted P-elements will be stable at their new locations. Transposition is predominantly limited to the germline and so the insertions are heritable. Therefore, P-elements can be used to randomly mutagenize the Drosophila genome, producing stable, heritable mutations.

In one preferred aspect, the same P-elements that are used to randomly mutagenize the genome also carry the reporter sequence. Preferably, the P-elements also comprise a marker gene allowing the inheritance of the P-elements to be correlated with the expression of the marker gene.

Also, preferably, the P-element being used as an insertable element does not itself encode transposase. For example, transposase function may be provided by an integrated P-element (e.g., such as the transposase source, P(ry⁺2-3) which is itself unable to hop from the genome or by a crippled P-element vector which is co-introduced with the mutagenizing P-element.

A DNA construct comprising a P-element, and preferably comprising a reporter sequence and marker gene, is injected into embryos of M strain females which lack P-elements and which do not express the marker gene. Suitable marker genes include those which provide a visible, easily selectable phenotype such as eye color, body color, wing morphology, and the like, as discussed above. In one aspect, the P-element comprises a mini-white gene whose expression in flies bearing the white mutation restores a red eye color to otherwise white-eyed flies. Suitable P-element vectors are described in, Pirrotta, et al. Vectors: A Survey of Molecular Cloning Vectors and Their Uses, edited by R. L. Rodriguez and D. T. Denhardt, Butterworths, Boston, 1988; and Rubin and Spradling, Nucleic Acids Res. 11(18): 6341-51, 1983, for example. In some aspects, enhancer or promoter trap vectors are used. For example, the P-element construct can comprise a promoter-less reporter gene sequence. Expression of the reporter gene sequence will only occur when the P-element construct is integrated downstream of a promoter and expression of the reporter gene will therefore reflect the transcription pattern of the modulator gene. Because the marker gene comprises a promoter, all insertion events will be detectable, not just the ones which bring the reporter gene in suitable proximity to the marker gene promoter. See, e.g., as described in Lucasovich, et al., Genetics 157: 727-742, 2001.

Microinjection is carried out using methods known in the art, such as described in Van Deusen, J. Embry. Exp. Morph. 37: 173, 1976. Typically, embryos are collected on lightly yeasted agar plates for one hour, then transferred to 17-18° C. Chorions are removed and embryos are aligned on double stick tape. Preferably, embryos are covered in oil (e.g., fluorocarbon oil) to minimize drying. Injections are performed at the posterior end of the embryo, since this end comprises the developing germ line cells of the fly.

After injection, embryos are maintained in a humidified chamber at 17-18° C. Hatched larvae are removed from the oil and placed on standard Drosophila cornmeal-molasses-yeast medium with subsequent development at 21-23° C.

Surviving embryos that develop into fertile adult flies are mated to non-M strains which also lack the marker gene. Progeny are examined to identify those flies that express the marker gene and therefore which include the P-element. Of these flies, a subset are crossed to flies bearing balancer chromosomes to prevent chromosomes bearing the P-element from recombining, to maintain stocks of flies bearing the mutant modulator genes, and to otherwise facilitate mapping of the P-element. Another subset is mated to other progeny in the subset to generate flies that are homozygous for the P-element.

Alternatively, M strain females are simply mated to males comprising a mutagenic P-element in their genome and expressing the 2-3 element, i.e., “jump-start” males.

Preferably, mutations are selected which result in the production of viable adult flies when homozygous for the P-element.

In yet another embodiment, flies from a stock center comprising P-element insertions may be crossed to l(2)gl flies and bred to produce flies that are homozygous for the P-element insertion and l(2)gl mutation. For example, flies from the Berkley Drosophila Genome Project (BDGP) Gene Disruption Project are available from the Bloomington Stock Center (Bloomington, Ind.) (see, e.g., Spradling, et al., Genetics 153: 135-177, 1999).

The use of P-elements in Drosophila is well-known in the art and is described in, for example, Rubin and Spradling, Science 218: 348-53, 1982; U.S. Pat. No. 4,670,388; Engels, Cold Spring Harbor Symp. 45: 561,1981.

Methods of fly husbandry are also routine in the art and described in, for example, in Ashburner, Fly Pushing: The Theory and Practice of D. melanogaster Genetics, Cold Spring Harbor Press, Plainview N.Y., 1977.

Identification of Modulator Mutations

Flies are bred which are homozygous both for the modulator mutation and the tumorigenic mutation and grown to larval stages using techniques well known in the art. See, Ashburner, 1977, supra. Cells from brain or imaginal discs are isolated for transplantation into adult flies that are wild type for both the modulator gene and tumorigenic gene and which do not express the reporter sequence. Cells or tissue fragments are then injected into the abdomens of female adult flies. Samples from greater than 100,000 different mutant lines may be examined in this way. Neoplastic tissues from flies homozygous for the tumorigenic gene and/or from flies which are wild-type for both the tumorigenic gene and modulator genes used as a control. Preferably, except for differences at the tumorigenic gene and modulator gene, the flies are otherwise genetically identical.

Alternatively, the host flies may be screened for modulator genes which affect the neoplastic phenotype of l(2)gl/l(2)gl tissues, by mutagenizing a non-l(2)gl background (e.g., with P-elements) and selecting for viable homozygous flies in which the establishment or metastasis of l(2)gl/l(2)gl neoplastic cells is altered, i.e., by transplanting cells from l(2)gl/l(2)gl larvae into adult flies homozygous for the modulator mutation. Such an assay may be used to screen for altered cell membrane receptors, extracellular matrix proteins and the like, that may be involved in the establishment or invasion of cancerous cells.

The tumorigenic and metastatic potential of these transplanted cells is evaluated by monitoring the expression of the reporter sequence in a plurality of cells in the adult fly. The assay used will generally depend on the nature of the reporter sequence selected. Preferably, the assay is one that can be performed in less than a day, and more preferably, can be performed in a few hours. Methods of detecting reporter gene expression in Drosophila are well known in the art. For example, Brandes, et al., describes detecting luciferase expression in Neuron 16: 687-692; Chalfie, et al., Science 263: 802-805

The plurality of cells is isolated from a variety of tissues types and/or body segments so that the impact of the modulator gene on cellular proliferation in the entire organism can be determined. Both tumorigenesis (i.e., numbers of flies with tumors in a population of flies; tumor size in an individual fly) and metastasis (number of tumors per fly and/or numbers of body segments/tissue types affected) can be monitored and quantified. In one aspect, cells from one or more of: the abdomen, thorax, head, wing and leg are obtained and the expression of the reporter sequence is determined and quantitated. In another aspect, whole body sections are isolated for immunohistochemistry or in situ hybridization analysis of reporter gene expression. Whole body immunohistochemistry may also be performed (i.e., without sectioning). A change in the numbers of different tissues expressing the marker gene and a change in the quantity of the marker gene product, in one or more tissues, identifies the presence of one or more mutated genes in the adult fly which are functional modulators of the neoplastic phenotype.

In one aspect, modulator genes are screened for which affect tumorigenesis and metastasis. In another aspect, modulator genes are screened for which affect tumorigenesis but not metastasis. In a further aspect, modulator genes are screened for which affect metastasis but not tumorigenesis.

Cloning of Associated Genes

Transposon-mediated mutagenesis such as mediated by P-elements, provides a useful way to map and clone modulator genes. P-element and/or reporter sequences can be used as probes in hybridization assays to cytogenetically map the site of the modulator mutation to a polytene chromosome band. For example, chromosomes can be prepared from larval salivary glands and hybridized in situ with a labeled probe. See, e.g., as described in Spradling Cell 27: 193, 1981.

The marker gene can be used in standard genetic assays (i.e., crosses) to map the modulator gene identified by P-element insertion. P-element sequences can be used to amplify sequences flanking an insertion site. For example, PCR can be performed using as primers, one or more of P-element sequences, the reporter sequence, and the marker sequence. See, e.g., Allen, et al. PCR Methods Appl. 4: 71-75. Amplified sequences flanking the P-element sequences can be sequenced using methods routine in the art and sequence information can be used to query a database of Drosophila sequences and/or sequences of other organisms (e.g., such as human beings).

Alternatively, or additionally, P-element sequences, reporter sequences, marker sequences, and/or amplified sequences may be used as hybridization probes to isolate genomic or cDNA clones from libraries derived from flies carrying the mutated modulator gene. Clones can be validated by cytogenetic analysis and/or mapping crosses. As an additional validation step, the ability of a clone to rescue the mutant modulator phenotype can be determined. In one aspect, modulator gene sequences are cloned into P-element vectors, and the ability of the sequences to rescue the modulator mutant phenotype is determined. Such vectors also provide the opportunity to increase the dose of the modulator gene product and to evaluate the affect of dosage on the neoplastic phenotype.

Preferably, cloned sequences are used to identify homologous sequences in human beings. For example, nucleic acid and protein sequences modulator genes can further be used as query sequences to perform a search against sequence databases to, for example, identify other family members or related sequences. Such searches can be performed using the NBLAST and XBLAST programs (version 2.0) of Altschul, et al. (J. Mol. Biol. 215: 403-10, 1990). BLAST nucleotide searches can be performed with the NBLAST program, with exemplary scores=100, and wordlengths=12 to obtain nucleotide sequences homologous to or with sufficient percent identity to the nucleic acid molecules of the invention. BLAST protein searches can be performed with the XBLAST program, with exemplary scores=50 and wordlengths=3 to obtain amino acid sequences sufficiently homologous to or with sufficient % identity (e.g., preferably, at least 60% identity). To obtain gapped alignments for comparison purposes, gapped BLAST can be used as described in Altschul, et al. (Nucleic Acids Res. 25(17): 3389-3402, 1997). When using BLAST and gapped BLAST programs, the default parameters of the respective programs (e.g., XBLAST and NBLAST) can be used.

The biological role of a cloned modulator gene is evaluated is methods known in the art. In one aspect, the expression of the gene is determined (e.g., by Northern, dot blotting, RT-PCR, in situ hybridization, immunoassays, and the like). In another aspect, the interaction of the modulator gene with one or more members of a molecular pathway is determined. Preferably, the molecular pathway is a signaling pathway. For example, in one preferred embodiment, larval brain extracts from flies homozygous for the modulator mutation and wild type or homo- or heterozygous for the l(2)gl mutation are arrayed onto a suitable substrate (e.g., such as a nitrocellulose slide) and the expression of a plurality of different pathway molecules in these extracts is determined using antibodies to modified (e.g., phosphorylated) and/or unmodified pathway molecules. Suitable pathway molecules whose expression can be evaluated include, but are not limited to: expression products of P13K; T-ERK; SMAD; P-SMAD1; Akt; Mad; cleaved caspase 3; Decapentaplegic (Dpp); l(2)gl genes, and the like. Arrays of nucleic acid samples can similarly be evaluated to monitor gene expression (e.g., in RT-PCR assays).

Identification of Modulator Compounds

In a different aspect, a method according to the invention comprises obtaining neoplastic tissue obtained from larvae of a fly homozygous for a tumorigenic mutation and introducing cells or tissue into an adult fly comprising wild-type for the tumorigenic gene. A candidate modulator compound is introduced into the nutrient medium on which an adult fly (preferably, newly eclosed), or a larval form, feeds. The ability of the modulator to alter the pattern of tumor growth in the fly is assessed. Populations of flies (e.g., greater than 100,000) can be screened in this way to identify candidate agents that affect tumor growth. The proliferation of neoplastic cells can be tracked by monitoring the expression of a reporter sequence inserted into the genome of such cells and assaying various segments of the adult fly for the presence of, levels of, and/or activity of, the reporter. This assay allows for quantitative and qualitative measures of abnormal cell proliferation in the flies being screened.

As above, both tumorigenesis and metastasis can be monitored and quantified. In one aspect, cells from one or more of: the abdomen, thorax, head, wing and leg are obtained and the expression of the reporter sequence is determined and quantitated. In another aspect, whole body sections are isolated for immunohistochemistry or in situ hybridization analysis of reporter gene expression. Alternatively, whole mounts can be evaluated. A change in the numbers of different tissues expressing the marker gene and a change in the quantity of the marker gene product, in one or more tissues, identifies the presence of one or more candidate modulator compounds in the adult fly which are functional modulators of the neoplastic phenotype.

Alternatively, one or more cells comprising a neoplastic phenotype may be transplanted into adult flies that have been fed, and/or are being fed different compounds to be assayed. The effect of the compounds on the induction and invasion of tumors is monitored generally as described above. In yet another aspect, the one or more cells are transplanted into adult flies and the adult flies are exposed to compound after transplantation.

These types of HTS assays also allow for a determination of the general toxicity of modulator compounds through 50% lethal dose (LD₅₀) computations.

Because large numbers of compounds can be screened, in one aspect, the method comprises screening a compound library for a modulator of the tumorigenic gene. Compound libraries may be purchased commercially (e.g., such as LeadQuest™-libraries from Tripos (St. Louis, Mo.)) or may be synthesized using methods well known in the art. Compounds may be introduced into the nutrient media on which larvae or adult Drosophila feed and the affect of the compounds can be assayed for by performing the whole-organism based-screening assay described above. Compounds may be delivered to individual flies or to groups of flies.

Suitable compound which can be tested, include, but are not limited to, carbohydrates, polyalcohols (e. g., ethylene glycol and glycerol), polyphenols (e.g., hydroquinones and tetracylines), small molecules, drugs, proteins, peptides, or pharmacophores thereof, peptoids, peptidomimetics, nucleic acids, nucleosides, metabolites, nucleic acid aptamers, protein aptamers, and the like. Compounds may be based on (i.e., pharmacophores of) naturally occurring extracellular or intracellular signaling molecules or their derivatives or the like. Compounds may be provided in a delivery vehicle such as a sucrose solution or in a liposome formulation.

In one aspect, eggs of the suitable genotype are collected on a nylon mesh and placed onto standard fly food. Approximately three to five day old larvae (third larval instar) are then collected and placed in suitable containers such as multiwell culture dishes comprising wells with a nutrient layer (e.g., such as agar supplemented with yeast) or in individual culture dishes. Compounds are either present in, or added to, the nutrient layer. Compounds may be provided to different larvae or sets of larvae at different doses. Delivery of compounds can be automated using an automated injection robot. Individual containers for larvae and or flies may be tagged using means known in the art such as bar code labels or radiofrequency tags.

Following a suitable exposure period, one or more cells from each larva (or sets of larvae) exposed to a particular compound are obtained and introduced into an adult fly to evaluate the neoplastic potential of the cells. Cells from different tissues are evaluated to survey the organism, e.g., samples can be obtained from the head, thorax, abdomen, leg, etc. to survey the expression of the reporter sequence. In one aspect, the level of reporter gene expression and/or spread of reporter gene expression is monitored. Where multiple flies are used to test particular compounds, tumor incidence in a plurality of flies can be determined. In still another aspect, the effect of different doses of compounds can be evaluated.

In one aspect, the HTS assay system is used to identify modulator compounds which ameliorate or eliminate a neoplastic phenotype. However, the system may also be used to assay determine the carcinogenic potential of known or unknown compounds. Because the biological affect of the compound on the entire organism is evaluated, more biologically relevant compounds should be identified than in cell-based screening assays.

Kits

The invention further provides compositions and kits. In one aspect, a kit comprises an array comprising a substrate, such as a nitrocellulose slide, glass, silicon, and the like. Substrates can be rigid (e.g., such as glass slides) or flexible, or semi-flexible (e.g., such as membranes). Samples comprising a plurality of different cellular polypeptides and/or nucleic acids from a mutant fly comprising a mutated modulator gene, identified as described above, are arrayed at different locations on the substrate (e.g., using an automatic microarrayer as described above).

In one aspect, the samples comprise extracts from one or more cells from larvae of the mutant fly strain. The fly strain comprising the mutated modulator gene may also be mutated for one or more copies of a tumorigenic gene. Tumorigenic genes include, but are not limited to l(2)gl, brat, (l(3)bt), l(3)mbt, Dlg, tu (2)-K, and e(tu-K). The fly strain may be heterozygous, homozygous, or hemizygous for the mutated modulator gene. Alternatively, the fly strain may be wild type with respect to modulator genes but may be heterozygous, hemizygous, or homozygous for the mutated tumorigenic gene.

Control samples may also be included in the array, such as samples from wild type flies and/or samples from organisms that are not flies (e.g. such as plant cell samples, and the like). Combinations of samples such as described above may be included in the arrays and variations of these arrays are obvious and are encompassed within the scope of the invention.

In one aspect, the kit comprises an array and at least one molecular probe. The molecular probe may be an antibody and/or a nucleic acid, or more generally, a binding partner with binding specificity for a cellular biomolecule (e.g., the probe may also be a nucleic acid or protein aptamer). Preferably, the probe is labeled. Multiple different types of probes may be included in the kit and these may be differentially labeled.

In one preferred aspect, the probe specifically binds to a molecular pathway molecule, such as a cell signaling protein. In another aspect, the kit comprises a plurality of probes specifically recognizing different molecules in the same pathway. In a further aspect, at least one probe in the kit recognizes a modified form of a polypeptide but does not recognize an unmodified form.

The invention also provides a composition comprising one or more isolated neoplastic cells from Drosophila. In one aspect, the composition comprises one or more cells comprising a mutation in a tumorigenic gene and expressing a tumorigenic phenotype (e.g., the cells are homozygous or hemizygous for a recessive mutation, or are heterozygous or homozygous for a dominant mutation). Tumorigenic genes include, but are not limited to, l(2)gl, brat, (l(3)bt), l(3)mbt, Dlg, tu (2)-K, and e(tu-K).

Preferably, the one or more cells are from one or more larvae. Also, preferably, the one or more cells comprise a reporter sequence. The reporter sequence may be selected from any of the sequences described above. Preferably, the reporter sequence is comprised within a P-element.

In one aspect, the one or more cells are frozen.

In another aspect, the invention provides a kit comprising one or more of the compositions described above and one or more reagents for facilitating injection of the one or more cells into an adult fly.

The one or more cells additionally may comprise at least one mutation in a modulator gene.

### EXAMPLES

The invention will now be further illustrated with reference to the following examples. It will be appreciated that what follows is by way of example only and that modifications to detail may be made while still falling within the scope of the invention.

### Example 1

Flies were reared in shell vials on standard cornmeal, molasses, and yeast medium at 20° C. Second chromosome lethal mutations were maintained over balancers marked with y⁺ and CyO mutations in stocks that were homozygous for the y mutation on the X-chromosome. Mutant larvae could be identified on the basis of expression of the y mutant phenotype.

Generation of Homozygous Mutations that Disrupt Metastasis

P-element insertion mutations were generated in a l(2)gl heterozygous background. A PlacWP-element inserted on the X chromosome was randomly mobilized in a heterozygous lethal giant larvae background by combination with the ‘jumpstarter’ P-element strain P(ry+; 02-3). Autosomal insertions were mapped by standard genetic methods using a yw/yw;+/+;+/+ stock and examining the segregation of CyO and the w⁺marker. A homozygous P-element stock was established from each independent insertion.

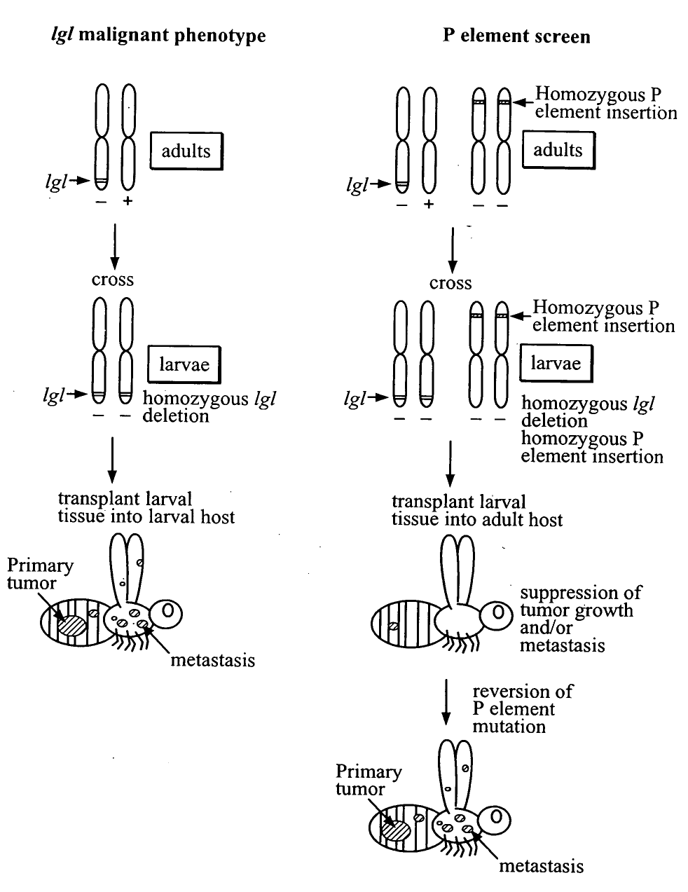

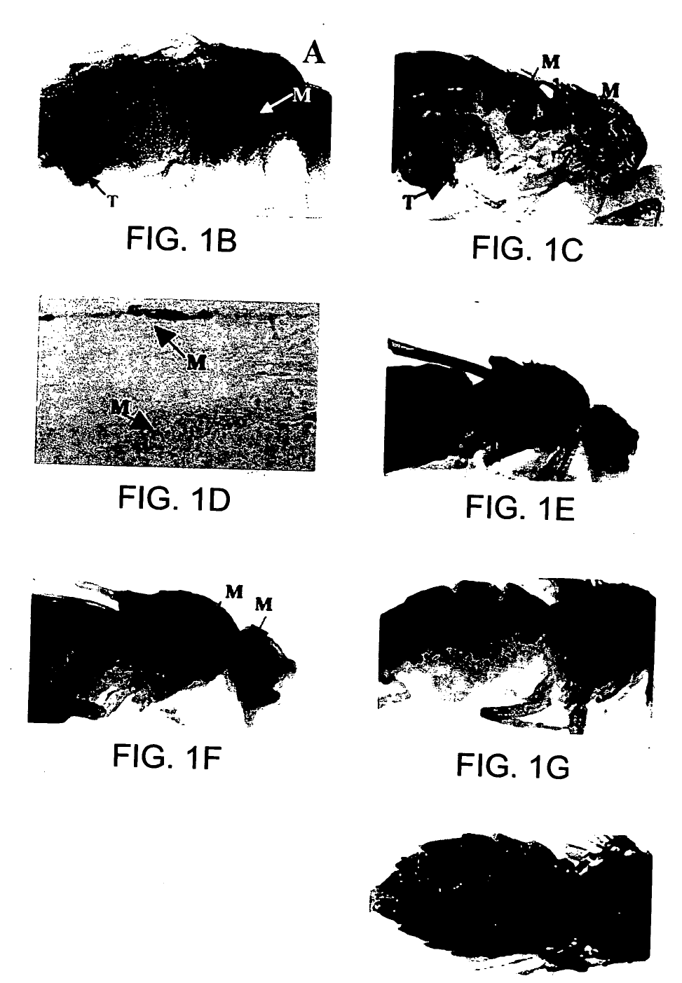

Homozygous l(2)gl larvae were isolated from P-element lines carrying two copies of the P-element insertion (FIG. 1A). Over 124,000 flies heterozygous for l(2)gl were screened for transposition of a single P-element originally on the X-chromosome. The mini-white gene was used as a marker to follow inheritance of the P-element via eye color. Nine hundred and eighty-six P-element insertions were isolated and mapped in this way. A line was established for each insertion carrying both copies of the P-element. In some cases, homozygosity of the P-element caused embryonic or early larval lethality. Mutations were selected which were homozygous viable.

Brain lobes from armadillo-lacZ marked larvae; were dissected and cut into halves. Each fragment was injected into the abdomen of a βgal^{ml} (a mutant that lacks endogenous 6-galactosidase expression) adult female using a 33 gauge needle, where it was cultured for 21 days at room temperature. Hosts that had received transplants were opened along the ventral midline and fixed in 3.7% formaldehyde in phosphate buffered saline (PBS). β-galactosidase in the donor tissue was detected by staining overnight at 37° C. in 0.02% X-gal in 10 mM Na pyrophosphate, 0.15 M NaCl, 1.0 MM MgCl₂, 5 mM ferricyanide, and 5 mM K ferrocyanide (Specialty Media). Secondary tumors were defined as β-galactosidase-marked cells distinct from the :implanted tumor, which was defined as the primary tumor.

When l(2)gl brain fragments were injected into adult hosts (see, arrowhead in FIG. 1B), the injected tissue proliferated as a primary tumor (T) and invaded adjacent tissue. Cells migrated away from the primary tumor to generate widespread metastatic colonies (M) (FIG. 1B-D). As previously described for the l(2)gl phenotype (Woodhouse, et al., 1998, supra), metastatic colonies are found in the abdomen (57%), thorax (70%), head (39%), wing (35%) and leg (48%). FIG. 1D shows a tissue section of invasive l(2)gl/l(2)gl tumors in host thorax muscle.

Homozygous P-element induced mutations were identified in which this phenotype was disrupted. Excision of the P-element was shown to restore the neoplastic phenotype. Tumorigenic and metastatic cells were visualized by lacZ staining after 21 days. FIG. 1E shows the neoplastic phenotype of the 97-2 insertion l(2)gl/97-2 insertion l(2)gl line. A primary tumor (T) is observed but no metastasis. FIG. 1F shows reversion back to a neoplastic phenotype in 97-2 excision l(2)gl/97-2 excision l(2)gl flies. FIG. 1G shows suppression of the neoplastic phenotype in the l(2)gl/l(2)gl; 23-2 insertion/23-2 insertion line. Tumorigenesis and metastasis is suppressed. FIG. 1H shows reversion to a metastatic phenotype in l(2)gl/l(2)gl; 23-2 excision/23-2 excision flies.

Identification of Functional Mutations and Cloning of Associated Genes

Insertion 97-2 completely blocked metastasis although it did not inhibit primary tumor growth (FIG. 1E) (12/12 in each group). Excision of the P-element reverted this line to the full l(2)gl metastatic phenotype (12/12) (FIG. 1F). P-element insertion 115-1 accelerated the lethality of injected tumors (12/12 in each group). When l(2)gl tissue was transplanted into 12 hosts, one half of the hosts survived 36 days, compared to 24 days for 115-1/l(2)gl flies. Furthermore, all of the hosts injected with l(2)gl tissue died within 60 days compared to 42 days for 115-1/l(2)gl (P<0.01).

A third P-element insertion, line 23-2, disrupted both the tumorigenesis and metastasis pattern of l(2)gl brain tissue (12/12 in each group). Two copies of this P-element insertion completely blocked proliferation of the l(2)gl primary tumor (FIG. 1G) but did not alter viability of the larva or grossly modify l(2)gl brains, which are composed of overgrown tissues with loosely adherent cells. Excision of the P-element in line 23-2 resulted in reversion to a tumorigenic and metastatic phenotype (12/12) (FIG. 1H) (p<0.01). Thus, the gene disrupted in this line is required for the l(2)gl malignant phenotype.

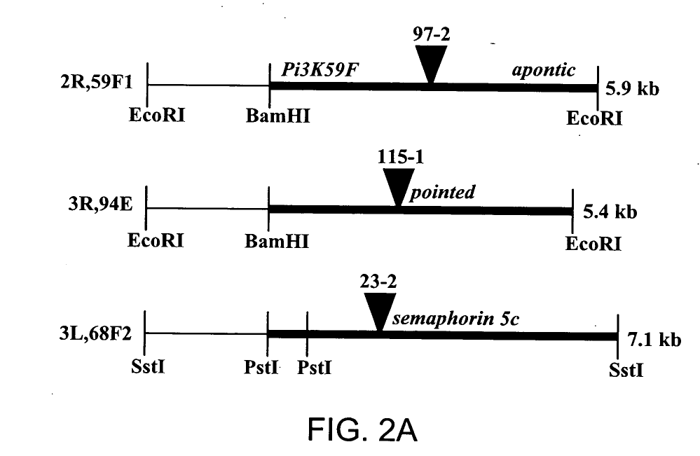

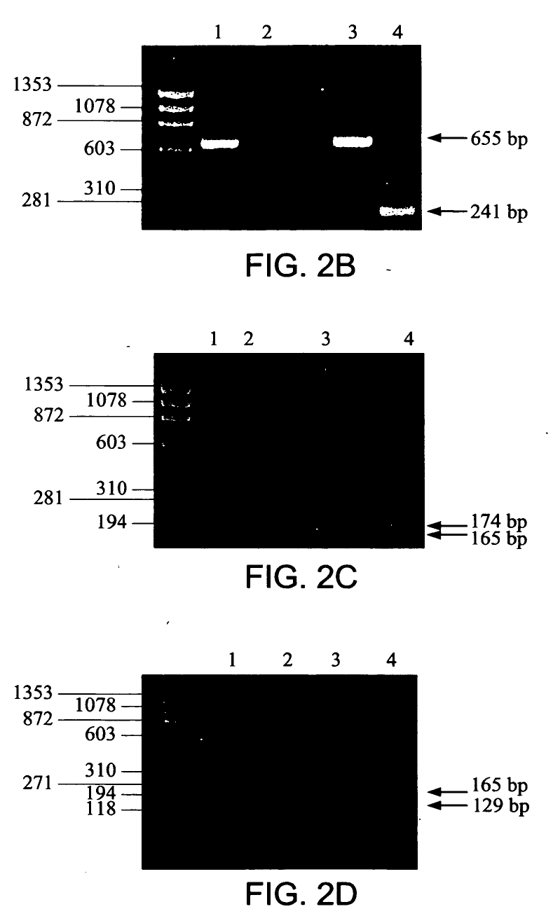

The genomic DNA at the 3′ end of the P-element was isolated by plasmid rescue (FIG. 2A) from adult Drosophila from each P-element line. The DNA was cut with a restriction enzyme and phenol-chloroform extracted. An EcoRi genomic fragment was isolated from lines 97-2 and 115-1 and an SstI genomic fragment was isolated from line 23-2. The fragments were ligated and phenol-chloroform extracted. One shot TOP 10 (Invitrogen) cells were transformed with the ligation mix. DNA was extracted from individual colonies and analyzed by restriction mapping using the second polylinker sites (BamHI for lines 97-2 and 115-1 and PstI for line 23-2). Cloned flanking sequences were sequenced at the NIH DNA minicore facility. Random hexamer-based reverse transcription was performed from third instar larvae total RNA.

The 97-2 insertion is on the right arm of the second chromosome at 68F2, between the Pi3K59F and apontic genes. The 115-1 insertion is on chromosome 3 at 94E in the pointed gene. The 23-2 P-element is inserted on the left arm of chromosome 3 at 68172 in the sema-5c gene. Confirmation of this localization was performed by PCR amplification of genomic DNA from each line with specific primers. One primer matched the P-element sequence near the 3′ end and the second primer matched a sequence in the flanking genomic DNA. PCR amplification with each insertion/P-element primer pair resulted in a product of a predicted size for that P-element line, but did not amplify a product in other lines including the parental line (see, e.g., FIG. 2B). PCR conditions were: 1 cycle 94° C. for 5 minutes, 35 cycles of 45 seconds at 94-C, 45 seconds at 58° C., 45 seconds 72° C., 1 cycle at 72° C.

P-Element Insertions caused Up-Regulation of Apontic and Pointed

Expression of the apontic and pointed genes were examined by RT-PCR in lines 97-2 and 115-1. The expression of apontic is present in the P-element line 97-2 and absent in the parental line (FIG. 2C). P13K was examined in the 97-2 line, as the insertion is between the Pi3K59F and apontic genes and could affect either or both genes. The protein expression levels of P13K in larval brains from the 97-2 insertion were not significantly altered. Thus, the inhibitory effect caused by the P-element insertion in line 97-2 on metastasis is due to the expression of apontic. The pointed gene was strongly up-regulated in the 115-1 insertion line compared to the E1 parental line (FIG. 2C). This caused increased host lethality of l(2)gl/l(2)gl,115-1/115-1 compared to l(2)gl/l(2)gl flies.

P-Element Insertion Causes Loss of SEMA5C Protein Expression

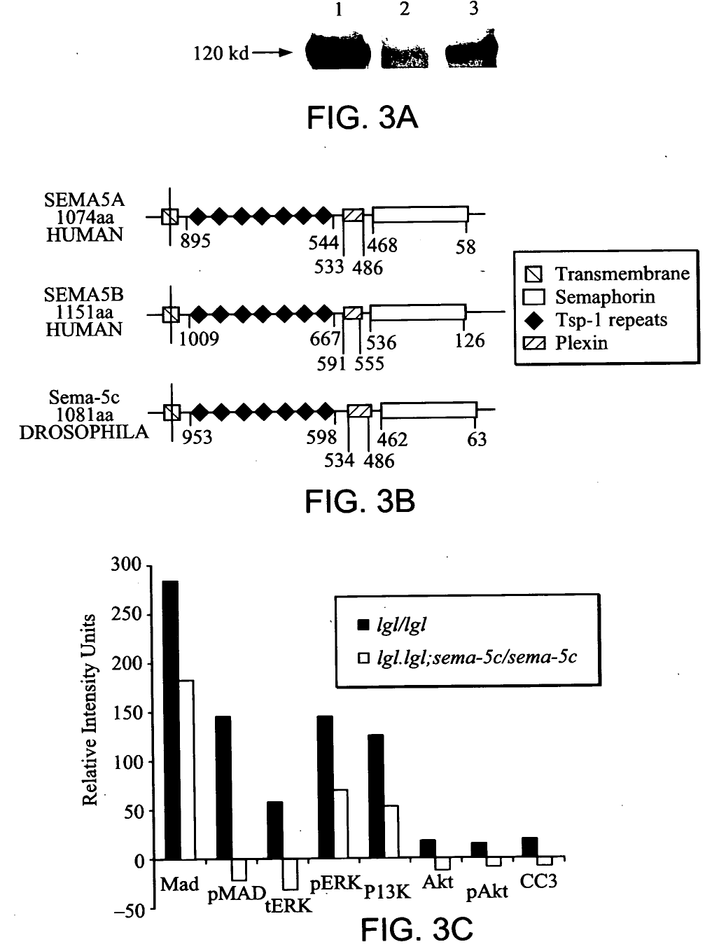

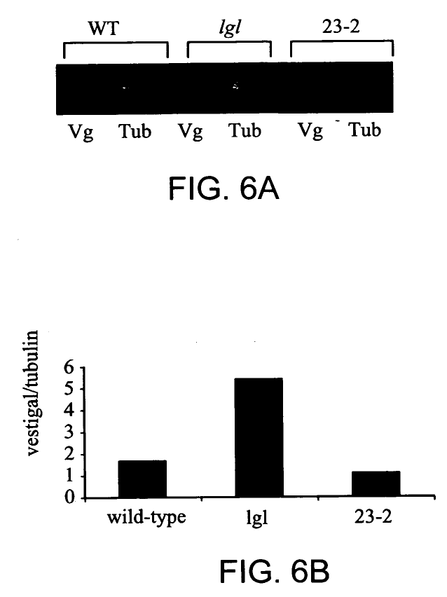

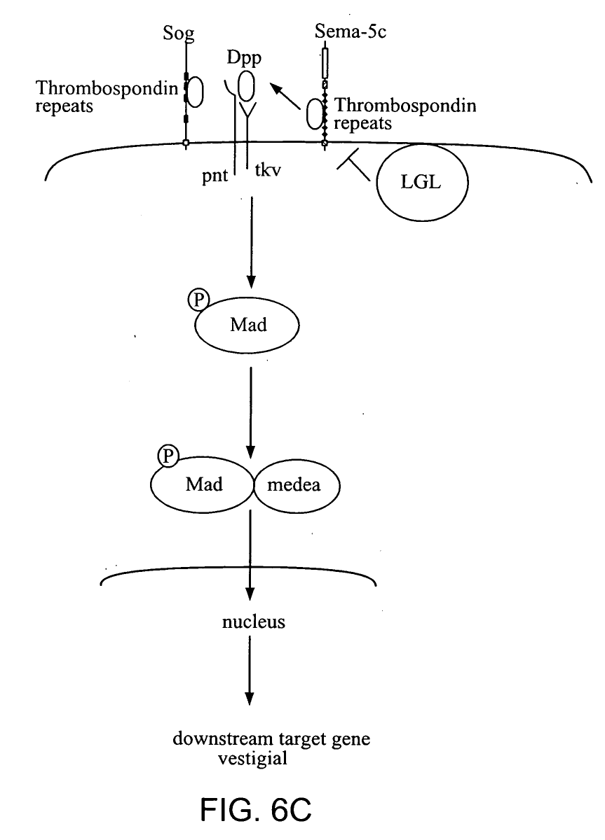

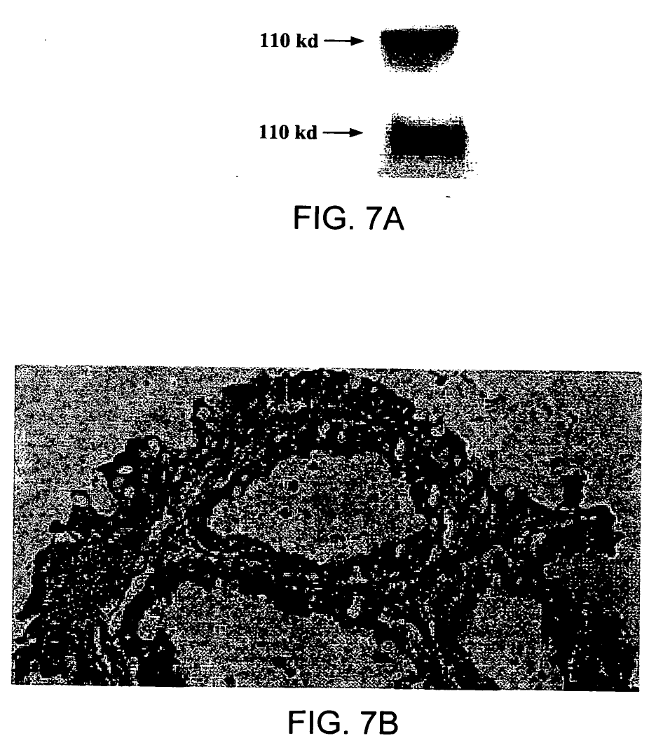

SEMA5C was undetectable in protein extracts from dissected brain tissue of homozygous 23-2 flies. Excision of the P-element resulted in recovery of protein expression (FIG. 3A) and restoration of the malignant phenotype (FIG. 1H). Based on sequence homology, two related mammalian semaphorins were identified with sequence domains similar to those of SEMA5C, SEMA5A and SEMA5B. All are class 5 semaphorins, containing thrombospondin repeats, a sema domain and a transmembrane domain (FIG. 3B). The SEMA5A and SEMA5D proteins were shown to be expressed in membrane preparations of A2058 cells (FIG. 7A) and MDA435 cells (data not shown). SEMA5D was shown by immunohistochemistry to be expressed in the membrane of ovarian cancer cells (FIG. 6B).

Up-Regulation of Murine and Human SEMA5A in Metastatic Cell Lines

The expression level of SEMA5A was studied in cell lines of varying metastatic potential. Larval brain extracts were prepared by dissection of brains from late third instar larvae and homogenization in RIPA buffer containing 500 μM AEBSF hydrochloride, 150 mM aprotinin, 1 μM E-64, 0.5 mM EDTA disodium, 1 μM leuptin hemisulfate. 2× Tris-Glycine. SDS sample buffer (Novex) with 4% β-mercaptoethanol was added and extracts were boiled 5 minutes. Cell line lysates were prepared in 25 μM HEPES, pH7.5, 150 MM NaCi, 1% Igepal CA-630, 10 mM MgCl₂, 1 mM EDTA, 2% glycerol, 500 μM AEBSF hydrochloride, 150 mM aprotinin, 1 μM E-64, 0.5 mM EDTA disodium, 1 μM leuptin hemisulfate.

Anti-peptide antibodies were generated and affinity purified against the sequence SVRIGLPKEESRN (SEQ ID NO. 1) in the plexin domain of the SEMA5C protein. Primary antibodies used were anti-P-SMAD1 (Cell Signaling) and anti-Tubulin 1:2000 (Sigma) antibodies. Binding was detected using ECL (Amersham) as is known in the art. To perform immunofluorescence, MDA435 cells were fixed in tissue culture dishes with 4% neutral buffered formaldehyde for 30 minutes. Stained cells were mounted in aqueous mounting media (DAKO).

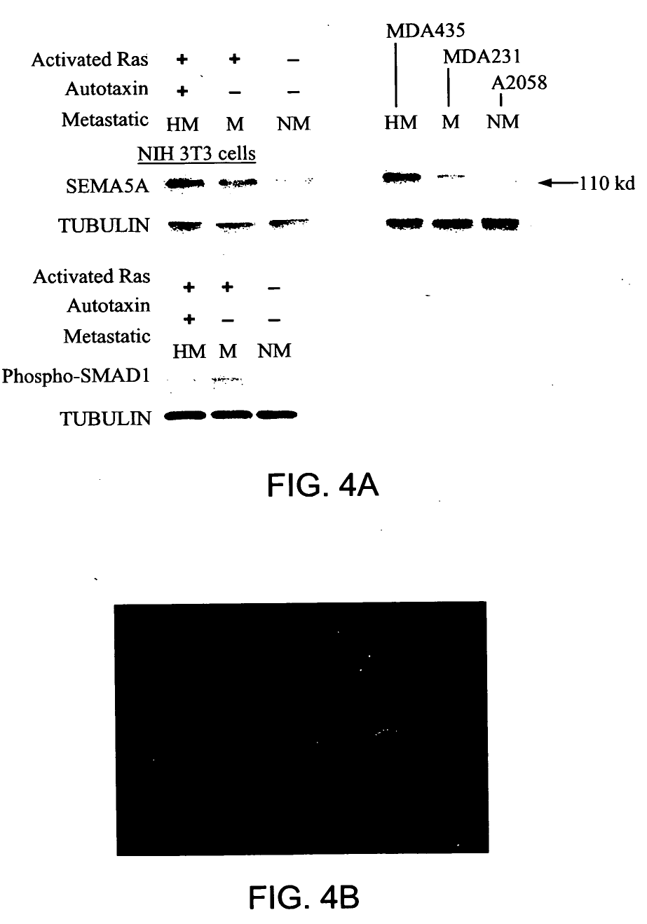

Western blot analysis using the anti-semaphorin antibody generated against the Drosophila SEMA5C revealed a single cross-reacting protein in murine as well as human cell lysates that corresponds in molecular weight to SEMA5A. SEMA5A expression was low in non-metastatic 3T3 cells, yet increased in metastatic Ras-transformed 3T3 cells (FIG. 4A).

The ATX gene has been shown to amplify the invasive and metastatic potential of Ras-transformed cells (Nam, et al., Oncogene 19: 241-247, 2001). SEMA5A was further elevated in the Ras+ATX-transformed 3T3 cells. SEMA5A expression was studied in human tumor lines of defined metastatic phenotype (Inoue, et al., J. Cell Physiol. 156: 212-217. 1993). The 3T3, 3T3-RAS, and 3T3-RAS-ATX cells were previously characterized (Nam et al., 2000, supra). Mice injected subcutaneously with 3T3 cells developed, on average, 3 lung metastases (range 0-16) while mice injected with 3T3-RAS-ATX developed, on average, 80 lung metastases (range 10-200) and those injected with untransfected 3T3 cells did not develop lung metastases (Nam et al., 2000, supra). Highly metastatic MDA435 expressed greater levels of semaphorin compared to low metastatic potential MDA231 or non-metastatic A2058 cells (FIG. 4A). Using immunofluorescence, the Semaphorin protein was localized to the cell membrane (FIG. 4B) in MDA435 cells.

Sema-5c Mutation Disrupts Dpp Signaling In l(2)gl Homozygotes

Selected signal transduction pathway phosphoproteins were examined by reverse phase protein microarray analysis (linearity r=0.99, s.d.<10% of the mean). See, e.g., Paweletz, et al., Oncogene 20: 1981-1989, 2001 (FIG. 3C). Larval brain extracts were prepared as described for Western blotting. A serial dilution of each lysate was prepared. A total of 50 nl (5 nl applied in a series of 10 separate applications) of the lysate was arrayed with a “pin and ring” GMS 417 microarrayer (Affymetrix) using a 500- micron pin onto nitrocellulose slides with a glass backing (Schleicher and Schuell). Spatial densities of 980 spots/slide were achieved on a 20 mm×50 mm slide.

Staining was performed using a DAKO Immunostainer automated slide stainer using the Catalyzed Signal Amplification (CSA) system (Dako) as previously described (Paweletz et al 2001). Antibodies used were: anti-actin 1:250 (Oncogene), anti-PI3K 1:100 (Cell Signaling), anti-T-ERK 1:500 (Cell Signaling), anti-P-ERK 1:1000 (Cell Signaling), anti-c-caspase 3 1:500 (Cell Signaling), anti-SMAD1 1:100 (Santa Cruz Biotechnology), and anti-P-SMAD1 1:250 (Cell Signaling). Cross reaction of the anti-human P-SMAD1 to Drosophila phospho-Mad was verified by treatment of disaggregated fly cells with 40 μg/ml dpp protein (R&D Systems). Specificity of each antibody was validated by detecting a single band by Western blotting. Arrays were scanned with an Epson Perfection 1640SU scanner using Adobe PhotoShop 5.5 at a resolution of 1200 dpi and analyzed with ImageQuant (Molecular Dynamics).

The levels of Mothers against dpp (Mad), P13K, ERK, Akt, and cleaved caspase 3 were studied in brain extracts from l(2)gl/l(2)gl, l(2)gl/l(2)gl; sema-5c/sema-5c, and wild-type larvae. P13K was reduced in l(2)gl/l(2)gl;sema-5c/sema-5c compared to l(2)gl/l(2)gl. To further study the role of P13K in l(2)gl tumors, the P13K inhibitor, LY294002, was orally administered to Drosophila adults injected with l(2)gl/l(2)gl tissue. LY294002 treatment reduced the primary tumor size to 7% of untreated hosts, without adverse effects to the hosts (data not shown).

The largest difference observed by protein microarray analysis between l(2)gl/l(2)gl and l(2)gl/l(2)gl; sema-5c/sema-5c larval brain protein extracts was in levels of phospho-Mad. P-Mad was overexpressed in l(2)gl/l(2)gl compared to wild-type tissues. Following loss of sema-5c, P-Mad levels were reduced below the wild-type.

The expression of genes downstream of phosph-Mad was examined by RT-PCR to characterize targets of Dpp that may play a role in l(2)gl phenotype. Genes identified to be regulated through Dpp signaling in the wing imaginal disk model are spalt and optomotor blind genes. The spalt and optomotor blind genes were unchanged in l(2)gl compared with wild type. The expression of vestigial was increases in l(2)gl tissue compared with wild-type or l(2)gl/l(2)gl; sema-5c/sema-5c mutants (FIG. 6 A-C).

Results

The l(2)gl tumor model exemplified above, combined with P-element mutagenesis, permits HTS screening for large numbers of mutations. Using the HTS assay system according to one aspect of the invention, three genes were identified that causally affect metastasis. Two of the genes, pointed and apontic, act at the level of regulation of gene transcription/translation and may influence multiple downstream genes. In contrast, the third gene, sema-5c, is a transmembrane protein at the cell surface, and may directly interact with other proteins outside the cell in a manner required for tumor growth and metastasis.

The mechanism by which loss of l(2)gl induces cancer in Drosophila has previously been completely unknown. Disruption of apontic specifically blocked metastasis but not tumorigenicity of l(2)gl tumors. The apontic gene is described as a transcription factor affecting genes necessary for migration (Eulenberg and Schuh, EMBO J. 16: 7156-7165, 1997) or homeotic targets (Gellon, et al., Development 124: 3321-3331, 1997). The apontic gene has also been reported to be a translational repressor of oskar mRNA (Lie and Macdonald, Development 126: 1129-1138, 1999). Therefore, the assay has identified a potential role for apontic as acting via downstream targets to control migration, and invasion of l(2)gl tumor cells. The HTS assay also indicates a potential role for the pointed gene in regulating l(2)gl metastasis through the regulation of downstream genes. Disruption of this gene caused an acceleration of lethality to hosts transplanted with 191 tumors. The pointed gene is a member of the ets-like transcription factor family (Klambt, Development 117: 163-176, 1993), conserved between vertebrates and Drosophila (Abagli, et al., Mech. Dev. 59: 29-40, 1996). The c-Ets l protooncogene has been shown to regulate the expression of genes important in extracellular matrix remodeling and invasion including stromelysin-1 (Wasylyk, et al., EMBO J. 10: 1127-1134, 1991), collagenase-1 (Gutman and Wasylyk, EMBO J. 9: 2241-2246, 1990), and urokinase-type plasminogen activator (Nerlov, et al., Oncogene 6: 1583-1592, 1991).

The sema-5c gene is shown here for the first time to be absolutely required for growth and metastasis of l(2)gl tumors. The absence of sema-5c in the mutant line completely blocked tumorigenesis and metastasis and reversion of the mutation recovered the malignant phenotype. The expression of the sema-5c homolog, SEMA5A, correlated with metastatic potential in 3T3, Ras-3T3, and Ras-ATX 3T3 cell lines. SEMA5A levels also correlated with metastatic potential in human breast carcinoma and melanoma cell lines (FIG. 4A). This suggests that class 5 semaphorins may also play a role in mammalian tumorigenesis and metastasis.

### Example 2

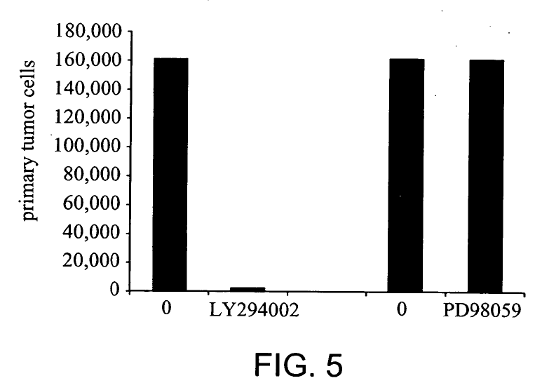

Adult βgal^{nl} hosts transplanted with armadillo-lacZ marked l(2)gl brain fragments were treated with 0; 0.556; 5.56; and 55.6 μg/ml of the PI-3 K inhibitor, LY294002 (Sigma), by adding drug to fly media. Flies were cultured for 21 days on drug-containing food and stained for the presence of β-galactosidase. Primary tumor size was determined by counting the cells dissociated from tumors. See, e.g., FIG. 5.

Variations, modifications, and other implementations of what is described herein will occur to those of ordinary skill in the art without departing from the spirit and scope of the invention and claims.

All of the patents, patent applications, international applications, and references described above are incorporated by reference herein in their entireties.

1. A method for identifying a modulator of a neoplastic phenotype comprising:
introducing a neoplastic tissue into an adult fly, wherein the neoplastic tissue comprises a reporter sequence;
exposing the fly to a candidate modulator;
monitoring the expression of the reporter sequence in cells from different tissues in the fly after the exposing,
wherein one or more of: a change in numbers of different tissues expressing the reporter sequence and a change in the level of reporter sequence expressed in one or more tissues, identifies the candidate modulator as a modulator of a neoplastic phenotype.

2. A method for identifying a mutated gene which is a modulator of a neoplastic phenotype, comprising:
introducing a neoplastic tissue into an adult fly, wherein the neoplastic tissue comprises a reporter sequence and is derived from a mutant fly comprising a mutated tumorigenic gene and wherein the mutant fly comprises at least one other mutated gene;
monitoring the expression of the reporter sequence in cells from different tissues in the adult fly, wherein one or more of: a change in the numbers of different tissues expressing the reporter sequence and a change in the level of reporter sequence expressed in one or more tissues, identifies the at least one other mutated gene as a modulator of a neoplastic phenotype.

3. A method for identifying a mutated gene which is a modulator of a neoplastic phenotype, comprising:
introducing a neoplastic tissue into an adult fruit fly, wherein the neoplastic tissue comprises a reporter sequence and is derived from a mutant fly comprising a mutated Drosophila tumorigenic gene and wherein the mutant fly comprises at least one other mutated gene;
monitoring expression of the reporter sequence in cells from a plurality of different tissues in the adult fily, wherein one or more of: a change in numbers of different tissues expressing the reporter sequence and a change in the quantity of the reporter sequence in one or more tissues, identifies the at least one other mutated gene as a modulator of a neoplastic phenotype.

4. The method according to claim 1, wherein the neoplastic tissue is obtained from a fly carrying a tumorigenic mutation.

5. The method according to claim 4, wherein the neoplastic tissue is obtained from a fly carrying a tumorigenic mutation selected from the group consisting of l(2)gl, brat, (l(3)bt), l(3)mbt, Dlg, tu (2)-K, and e(tu-K).

6. The method according to claim 4, wherein the tumorigenic mutation comprises l(2)gl.

7. The method according to claim 4, wherein the mutation is an amorphic mutation.

8. The method according to claim 7, wherein the mutation is a null mutation.

9. The method according to claim 7, wherein the mutation comprises a deletion.

10. The method according to claim 4, wherein the mutation is a conditional mutation which causes abnormal cell proliferation under selected conditions.

11. The method according to claim 10, wherein the selected condition is temperature.

12. The method according to claim 4, wherein the neoplastic tissue is obtained from one or more larvae.

13. The method according to claim 12, wherein the neoplastic tissue is brain tissue or imaginal disc tissue.

14. The method according to claim 1, wherein the reporter sequence is comprised within a P-element.

15. The method according to claim 14, wherein the reporter sequence is selected from the group consisting of lacZ gene, GFP gene, BFP gene, and luciferase gene.

16. The method according to claim 1, wherein the modulator modulates one or more of tumorigenesis or metastasis.

17. The method according to claim 1, wherein the modulator suppresses one or more of tumorigenesis or metastasis.

18. The method according to claim 1, wherein the modulator modulates metastasis but not tumorigenesis.

19. The method according to claim 16, wherein average size of tumors is altered.

20. The method according to claim 2, wherein the mutant fly is homozygous for the at least one other mutated gene.

21. The method according to claim 2, wherein the at least one other mutated gene comprises a P-element insertion.

22. The method according to claim 2, wherein samples comprising a plurality of different cellular polypeptides and/or nucleic acids from the mutant fly are arrayed on a substrate and contacted with one or more probes for specifically identifying a molecular pathway molecule.

23. The method according to claim 22, wherein the molecule is a polypeptide.

24. The method according to claim 22, wherein the pathway is a cell signaling pathway.

25. The method according to claim 22, wherein the nucleic acids are expressed sequences.

26. The method according to claim 22, wherein the array comprises nucleic acids and the probe comprises a nucleic acid.

27. The method according to claim 22, wherein the array comprises polypeptides and the probe comprises an antibody.

28. The method according to claim 26, wherein the probe comprises a plurality of different nucleic acids.

29. The method according to claim 28, wherein the different nucleic acids are differentially labeled.

30. The method according to claim 29, wherein the different nucleic acids are contacted to different areas on the array.

31. The method according to claim 27, wherein the probe comprises a plurality of different antibodies.

32. The method according to claim 27, wherein the probe comprises an antibody which recognizes a modified form of a polypeptide but which does not recognize an unmodified form of the polypeptide.

33. The method according to claim 31, wherein the antibodies are differentially labeled.

34. The method according to claim 31, wherein the antibodies are contacted to different areas of the array.

35. The method according to claim 22, wherein cell extracts from the mutant fly are arrayed on the substrate.

36. The method according to claim 22, wherein the cell extracts are from a larval form of the mutant fly.

37. The method according to claim 22, wherein at least one location on the array comprises cellular polypeptide or nucleic acids from a fly comprising at least one mutation in a gene selected from the group consisting of: l(2)gl, brat, (l(3)bt), l(3)mbt, Dlg, tu (2)-K, and e(tu-K).

38. The method according to claim 22 or 37, wherein at least one location on the array comprises cellular polypeptides and/or nucleic acids from a wild type fly.

39. An array comprising a substrate, wherein samples comprising a plurality of different cellular polypeptides and/or nucleic acids from a mutant fly according to claim 2 are arrayed on the substrate.

40. The array according to claim 39, wherein at least one location on the array comprises cellular polypeptides and/or nucleic acids from a fly comprising at least one mutation in a gene selected from the group consisting of: l(2)gl, brat, (l(3)bt), l(3)mbt, Dlg, tu (2)-K, and e(tu-K).

41. A kit comprising an array according to claim 39 and at least one molecular probe.

42. The kit according to claim 41, wherein the at least one molecular probe is an antibody and/or nucleic acid.

43. The kit according to claim 41, wherein the probe is a nucleic acid.

44. The kit according to claim 41, wherein the probe specifically binds to a molecular pathway molecule.

45. The kit according to claim 44, wherein the molecular pathway molecule is a cell signaling molecule.

46. The kit according to claim 41, comprising a plurality of different molecular probes.

47. The kit according to claim 41, wherein the probe recognizes a modified form of a polypeptide but not an unmodified form.

48. A composition comprising one or more isolated cells from a Drosophila larvae comprising a mutation in a tumorigenic gene and expressing a tumorigenic phenotype and further comprising a reporter sequence.

49. The composition according to claim 48, wherein the reporter sequence comprises a lacZ gene, a GFP gene, a BFP gene, or a luciferase gene.

50. The composition according to claim 48, wherein the reporter sequence is comprised within a P-element.

51. The composition according to claim 48, wherein the one or more cells are homozygous or hemizygous for the mutation in the tumorigenic gene.

52. The composition according to claim 48, wherein the tumorigenic gene comprises l(2)gl.

53. The composition according to claim 48, wherein the one or more cells is frozen.

54. The composition according to claim 48, wherein the one or more cells comprises at least one mutation in a modulator gene

55. A kit comprising a composition according to claim 48, and one or more reagents for facilitating injection of the one or more cells into an adult fly.
